(19) United States
(12) Patent Application Publication
(10) Pub. No.: US 2025/0090075 A1
(43) Pub. Date: Mar. 20, 2025

(54) ECG SIGNAL RECONSTRUCTION FROM EEG SIGNAL

(71) Applicant: Apple Inc., Cupertino, CA (US)

(72) Inventors: Christopher M. Sandino, Menlo Park, CA (US); Ali Moin, San Mateo, CA (US); Erdrin Azemi, San Mateo, CA (US); Joseph Cheng, Cupertino, CA (US)

(21) Appl. No.: 18/884,061

(22) Filed: Sep. 12, 2024

Related U.S. Application Data
(60) Provisional application No. 63/539,316, filed on Sep. 19, 2023.

(51) Int. Cl. A61B 5/318

(52) U.S. Cl. CPC A61B 5/318

(57) ABSTRACT

Embodiments are disclosed for ECG signal reconstruction from an EEG signal. In some embodiments, a method comprises: determining, with at least one processor, heartbeat timestamps from a heartrate signal; obtaining an electroencephalography (EEG) signal from an EEG sensor; aligning, with the at least one processor, the EEG signal with the heartbeat timestamps; collaborative filtering, with the at least one processor, the beat-aligned EEG signal; and determining, with the at least one processor, an approximation of an electrocardiography (ECG) signal based on the filtered beat-aligned EEG signal.

### CROSS-REFERENCE TO RELATED APPLICATION

This application claims priority to U.S. Provisional Patent Application No. 63/539,316, filed Sep. 19, 2023, the entire contents of which are incorporated herein by reference.

### TECHNICAL FIELD

This disclosure relates generally to electrocardiography (ECG).

### BACKGROUND

An ECG records the electrical signals in the heart to detect heart problems and monitor a user's health. An ECG is often recorded in a doctor's office, a clinic, an ambulance, or an operating room. Some personal devices, such as smartwatches, offer ECG monitoring. ECG monitoring on a smartwatch typically requires an active input from the user (e.g., press finger to crown) to start recording. However, this approach can miss irregular rhythms recorded throughout the day when the user is not providing the input.

Some smartwatches use photoplethysmography (PPG) to passively detect irregular rhythms throughout the day without active input from the user. Pulse rate, e.g., can be measured using a PPG sensor (e.g., for 1 minute). If an irregular pulse is detected, the user is notified by an irregular rhythm notification (IRN). IRNs are strongly predictive of the presence of atrial fibrillation (AFib). IRNs are also predictive of other kinds of arrhythmias, such as premature ventricular contractions (PVC) and premature atrial contractions (PAC). However, these other kinds of arrhythmias typically require an ECG monitor (e.g., an ECG patch) to confirm.

### SUMMARY

Embodiments are disclosed for ECG signal reconstruction from an Electroencephalography (EEG) signal.

In some embodiments, a method comprises: determining, with at least one processor, heartbeat timestamps from a heartrate signal; obtaining an electroencephalography (EEG) signal from an EEG sensor; aligning, with the at least one processor, the EEG signal with the heartbeat timestamps; collaborative filtering, with the at least one processor, the beat-aligned EEG signal; and determining, with the at least one processor, an approximation of an electrocardiography (ECG) signal based the filtered beat-aligned EEG signal.

In some embodiments, the beat-aligned EEG signal is a two-dimensional (2D) matrix.

In some embodiments, the ECG signal is determined based on a low-dimensional matrix approximation of the filtered beat-aligned EEG matrix.

Other embodiments are directed to an apparatus, system, and computer-readable medium.

Particular embodiments described herein provide one or more of the following advantages. Passive ECG signals are reconstructed from EEG signals to help differentiate between different arrhythmia types.

### BRIEF DESCRIPTION OF THE DRAWINGS

FIG. 1 is a block diagram of a system that reconstructs an ECG signal from an EEG signal, according to one or more embodiments.

FIGS. 2A-2C are plots illustrating rank-4 reconstruction of an ECG signal from an EEG signal using 114 beats, according to one or more embodiments.

FIGS. 3A-3C are plots illustrating rank-1 reconstruction of an ECG signal from an EEG signal using 63 beats, according to one or more embodiments.

FIG. 4 is a blow up a portion of the reconstructed ECG signal and ground truth ECG signal in FIG. 3B, according to one or more embodiments.

FIG. 5 is a flow diagram of a process for, reconstructing an ECG signal from an EEG signal according to one or more embodiments.

FIG. 6 is a block diagram of a mobile device architecture for implementing the system, features and processes described in reference to FIGS. 1-5.

### DETAILED DESCRIPTION

### System Overview

Systems, methods, and computer-readable storage mediums are disclosed for reconstructing an ECG signal from an EEG signal captured by one or more EEG electrode sensors placed inside, for example, the ear canal. The one or more EEG electrode sensors measure voltage potential changes due to neural activity occurring in the temporal lobe in the brain of the user. The user's heart produces electrical pulses that travel from the user's chest upward towards the user's head. The signal-to-noise ratio (SNR) of these electrical pulses measured at the ear are lower than those measured at the chest due to the large distance from the ear to the heart. Additionally, electrodes placed at the ear are sensitive to EEG signals from the temporal lobe in the brain, causing a mixture of brain EEG and ECG signals. Because the ECG waveform is periodic, the disclosed embodiments use collaborative filtering to reconstruct the ECG signal from low SNR EEG measurements.

In some embodiments, a one-dimensional (1D) time-series representation of heart rate data measured by the heart rate sensor is used to determine heartbeat timestamps (the start and end of each heartbeat) of the user. The timestamps are used to align a “noisy” EEG signal to heartbeat timestamps and construct a 2D beat-aligned matrix where the rows of the matrix correspond to time inputs, and the columns of the matrix correspond to heartbeat timestamps. In some embodiments, the heart rate signal is measured using, e.g., one or more PPG sensors that are located on the user's body at any location that is suitable for PPG sensing (e.g., ear, head, wrist, etc.). In other embodiments, EEG signals are generated by EEG electrode sensors around the head or with a device (e.g., an audio ear bud) inserted in the ear canal, or around the ear of a user. In some embodiments, the heart rate signal can also be measured using an infrared sensor, PPG sensor, or a microphone placed within the ear, around the ear, or around the head, that is sensitive to small pulsations caused by blood vessels in the ear.

Once the heartbeat timestamps are extracted from the heart rate signal, and the 2D beat-aligned matrix is constructed, collaborative filtering is used to project the 2D matrix into a low-dimensional representation using, for example, singular value decomposition (SVD). In some embodiments, the low-dimensional representation includes a dictionary that describes the dominant modes across the columns (heartbeats) in the 2D matrix. In some embodiments, the dictionary is truncated using an empirically defined threshold, such that dictionary includes only the most dominant modes. The dictionary is re-expanded into its original 2D matrix representation containing the denoised data. The 2D matrix is then flattened into the original 1D time-series representation to obtain the reconstructed ECG signal.

In some embodiments, a neural network is used to extract the dominant modes (as opposed to SVD). If trained with true ECG data, the neural network can outperform the SVD approach, by learning to extract non-orthogonal dictionaries that are specifically tuned for ECG morphologies.

### Example System

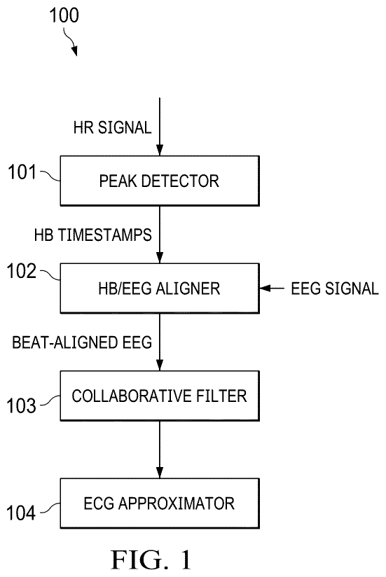

FIG. 1 is a block diagram of system 100 that reconstructs an ECG signal from an EEG signal, according to one or more embodiments. System 100 includes peak detector 101, HB/EEG aligner 102, collaborative filter 103, and ECG approximator 104.

A 1D time-series heartrate (HR) signal is input into peak detector 101 which generates heartbeat timestamps. The heartbeat timestamps are used by HB/EEG aligner 102 to align an EEG signal with the heartbeat timestamps. The EEG signal is captured by an EEG sensor. The EEG sensor can be inserted in the ear canal, around the ear, or located elsewhere on the user's head. For example, an audio earbud can include EEG electrode sensors that generate electrical signals in response to brainwaves. In some embodiments, the output of HR/EEG aligner 102 is a 2D beat-aligned matrix where the rows correspond to time points, and the columns correspond to heartbeat timestamps.

The beat-aligned EEG data is input into collaborative filter 103, which recovers underlying ECG data that is mixed with EEG signals arising from brainwaves, using matrix factorization or other collaborative filtering algorithm (e.g., deep learning).

ECG approximator 104 computes a rank-k matrix approximation of the ECG signal based on singular values of the beat-aligned matrix and the top singular vectors of the beat-aligned EEG matrix. For example, a rank-k approximation is given by SVD, where S is a nonnegative m×n diagonal matrix with diagonal entries sorted from high to low, and V^{H} is a n×n orthogonal matrix. If only the top k right and k left singular vectors are used (columns in U and V^{H}), then: Aₖ=Uₖ×Sₖ×Vₖ^{H}, where Aₖ is the beat-aligned 2D EEG matrix, where the “H” superscript is the Hermitian transpose. Finally, the 2D EEG matrix is flattened into the original 1D time-series representation to obtain the reconstructed ECG signal.

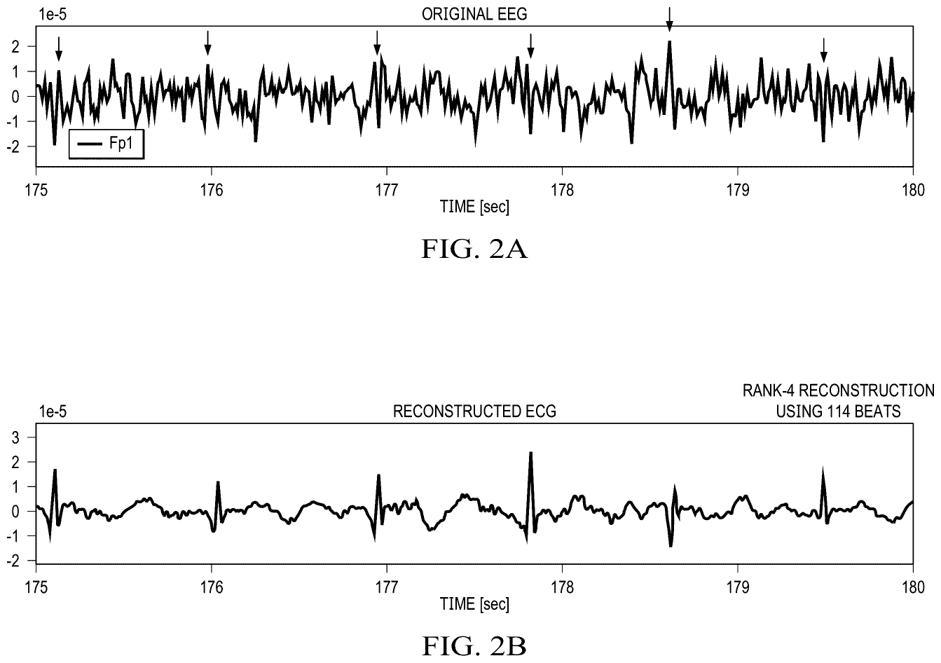

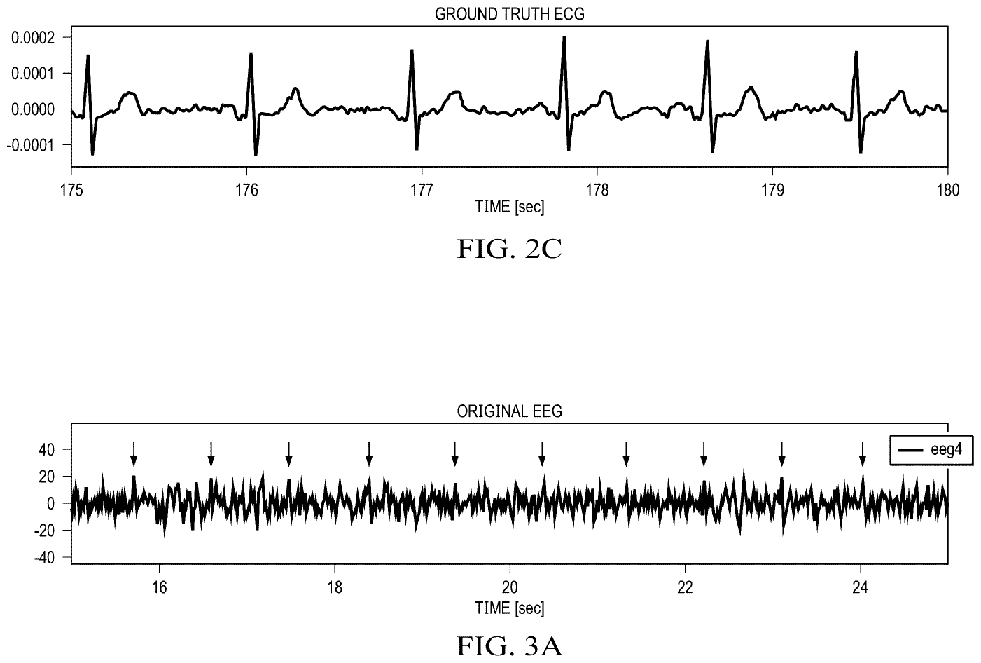

FIGS. 2A-2C are plots illustrating the results of system 100 for Cognionics data, according to one or more embodiments. In this example plot, a rank-4 approximation of the ECG signal using 114 heartbeats is shown. FIG. 2A shows the original EEG signal captured by an EEG sensor. FIG. 2B shows the reconstructed ECG signal. FIG. 2C shows a ground truth ECG signal captured by, e.g., an ECG patch. The arrows point to the locations of the heartbeats in the signals. Note that the rank-4 approximation of the ECG signal is a close approximation of the ECG ground truth signal.

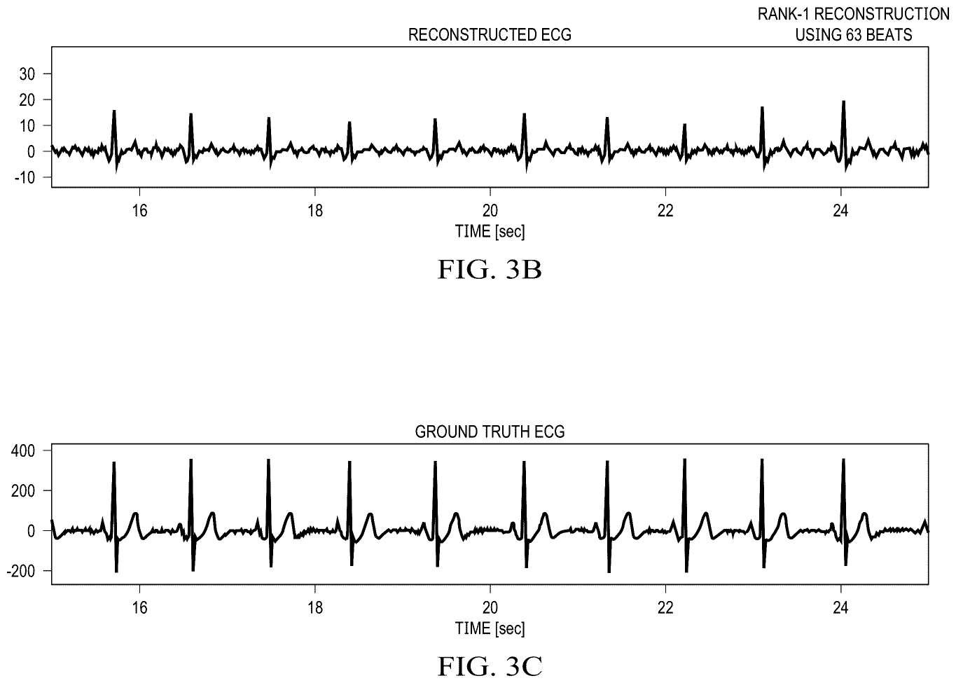

FIGS. 3A-3C are plots illustrating rank-1 approximation of an ECG signal from an EEG signal using 63 beats, according to one or more embodiments. FIG. 3A shows the original EEG signal captured by the EEG sensor. FIG. 3B shows the rank-1 approximation of the ECG signal. FIG. 3C shows a ground truth ECG signal captured by, e.g., an ECG patch. The arrows point to the locations of the heartbeats in the signals. Note that the rank-1 approximation of the ECG signal is a close approximation of the ground truth ECG signal.

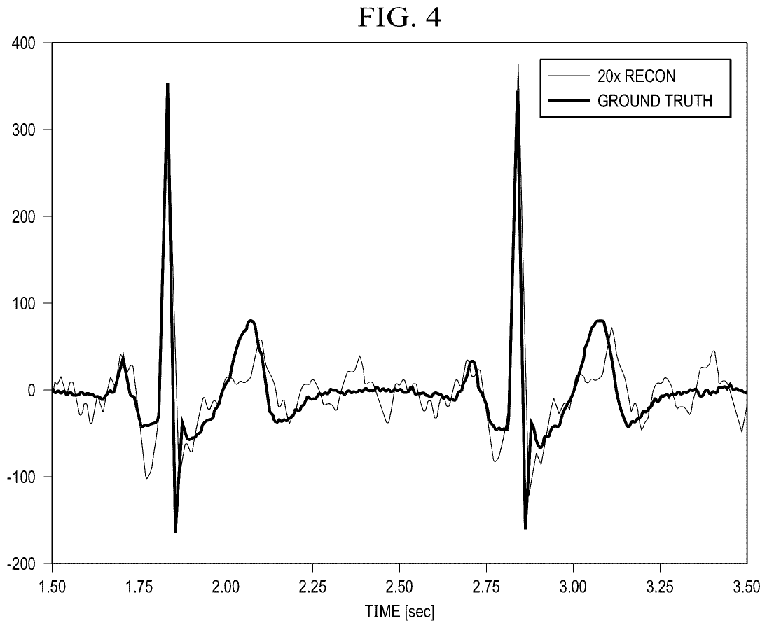

FIG. 4 is a blow up of a portion of the approximated ECG signal and ground truth ECG signal in FIG. 3B, according to one or more embodiments. A shown, the rank-1 approximation of the ECG signal is a close approximation of the ECG ground truth signal.

### Example Processes

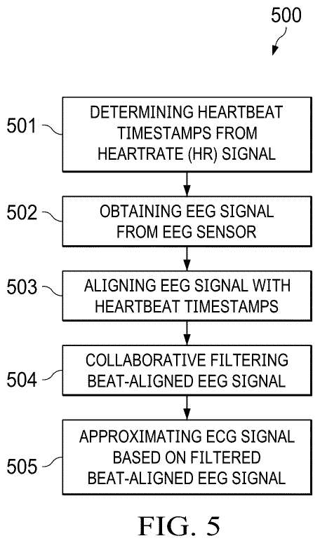

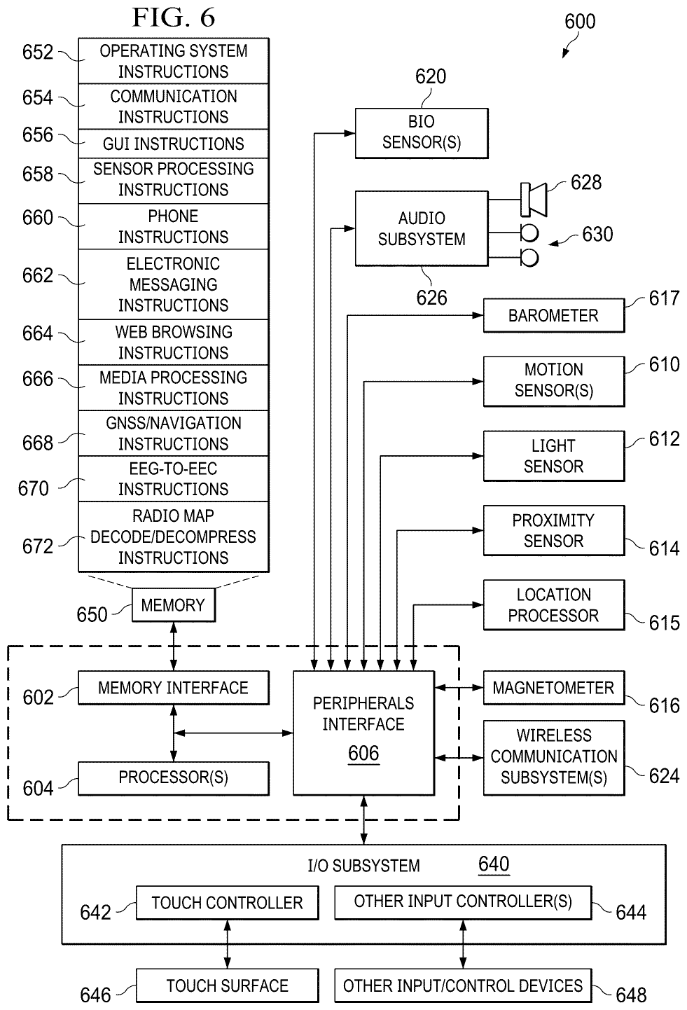

FIG. 5 is a flow diagram of a process 500 for, reconstructing an ECG signal from an EEG signal according to one or more embodiments. Process 500 can be implemented using, e.g., the architecture shown in FIG. 6.

Process 500 includes: determining heartbeat timestamps from a heartrate (HR) signal (501), obtaining an EEG signal (502) from an EEG sensor, aligning the EEG data with the heartbeat timestamps (503), collaborative filtering the beat-aligned EEG signal (504); determining an approximation of ECG signal based on the filtered beat-aligned EEG signal (505).

### Example Device Architecture

FIG. 6 is a block diagram of a device architecture for implementing the features and processes described in reference to FIGS. 1-5. Architecture 600 can include memory interface 602, one or more hardware data processors, image processors and/or processors 604 and peripherals interface 606. Memory interface 602, one or more processors 604 and/or peripherals interface 606 can be separate components or can be integrated in one or more integrated circuits. System architecture 600 can be included in any suitable electronic device for crash detection, including but not limited to: a smartwatch, smartphone, fitness band and any other device that can be attached, worn, or held by a user.

Sensors, devices, and subsystems can be coupled to peripherals interface 606 to provide multiple functionalities. For example, one or more motion sensors 610, light sensor 612 and proximity sensor 614 can be coupled to peripherals interface 606 to facilitate motion sensing (e.g., acceleration, rotation rates), lighting and proximity functions of the wearable device. Location processor 615 can be connected to peripherals interface 606 to provide geo-positioning. In some implementations, location processor 615 can be a GNSS receiver, such as the Global Positioning System (GPS) receiver. Electronic magnetometer 616 (e.g., an integrated circuit chip) can also be connected to peripherals interface 606 to provide data that can be used to determine the direction of magnetic North. Electronic magnetometer 616 can provide data to an electronic compass application. Motion sensor(s) 610 can include one or more accelerometers and/or gyros configured to determine change of speed and direction of movement. Barometer 617 can be configured to measure atmospheric pressure (e.g., pressure change inside a vehicle). Bio signal sensor 620 can be one or more of a PPG sensor, an electroencephalogram (EEG) sensor, an electrocardiogram (ECG) sensor, an electromyogram (EMG) sensor, a mechanomyogram (MMG) sensor (e.g., piezo resistive sensor) for measuring muscle activity/contractions, an electrooculography (EOG) sensor, a galvanic skin response (GSR) sensor, a magnetoencephalogram (MEG) sensor and/or other suitable sensor(s) configured to measure bio signals.

Communication functions can be facilitated through wireless communication subsystems 624, which can include radio frequency (RF) receivers and transmitters (or transceivers) and/or optical (e.g., infrared) receivers and transmitters. The specific design and implementation of the communication subsystem 624 can depend on the communication network(s) over which a mobile device is intended to operate. For example, architecture 600 can include communication subsystems 624 designed to operate over a GSM network, a GPRS network, an EDGE network, a Wi-Fi™ network and a Bluetooth™ network. In particular, the wireless communication subsystems 624 can include hosting protocols, such that the crash device can be configured as a base station for other wireless devices.

Audio subsystem 626 can be coupled to a speaker 628 and a microphone 30 to facilitate voice-enabled functions, such as voice recognition, voice replication, digital recording, and telephony functions. Audio subsystem 626 can be configured to receive voice commands from the user. Audio subsystem 626 can be used to capture audio during a crash and to convert the audio to SPL for crash detection processing.

I/O subsystem 640 can include touch surface controller 642 and/or other input controller(s) 644. Touch surface controller 642 can be coupled to a touch surface 646. Touch surface 646 and touch surface controller 642 can, for example, detect contact and movement or break thereof using any of a plurality of touch sensitivity technologies, including but not limited to capacitive, resistive, infrared, and surface acoustic wave technologies, as well as other proximity sensor arrays or other elements for determining one or more points of contact with touch surface 646. Touch surface 646 can include, for example, a touch screen or the digital crown of a smart watch. I/O subsystem 640 can include a haptic engine or device for providing haptic feedback (e.g., vibration) in response to commands from processor 604. In an embodiment, touch surface 646 can be a pressure-sensitive surface.

Other input controller(s) 644 can be coupled to other input/control devices 648, such as one or more buttons, rocker switches, thumbwheel, infrared port, and USB port. The one or more buttons (not shown) can include an up/down button for volume control of speaker 628 and/or microphone 630. Touch surface 646 or other controllers 644 (e.g., a button) can include, or be coupled to, fingerprint identification circuitry for use with a fingerprint authentication application to authenticate a user based on their fingerprint(s).

In one implementation, a pressing of the button for a first duration may disengage a lock of the touch surface 646; and a pressing of the button for a second duration that is longer than the first duration may turn power to the mobile device on or off. The user may be able to customize a functionality of one or more of the buttons. The touch surface 646 can, for example, also be used to implement virtual or soft buttons.

In some implementations, the mobile device can present recorded audio and/or video files, such as MP3, AAC and MPEG files. In some implementations, the mobile device can include the functionality of an MP3 player. Other input/output and control devices can also be used.

Memory interface 602 can be coupled to memory 650. Memory 650 can include high-speed random-access memory and/or non-volatile memory, such as one or more magnetic disk storage devices, one or more optical storage devices and/or flash memory (e.g., NAND, NOR). Memory 650 can store operating system 652, such as the iOS operating system developed by Apple Inc. of Cupertino, California. Operating system 652 may include instructions for handling basic system services and for performing hardware dependent tasks. In some implementations, operating system 652 can include a kernel (e.g., UNIX kernel).

Memory 650 may also store communication instructions 654 to facilitate communicating with one or more additional devices, one or more computers and/or one or more servers, such as, for example, instructions for implementing a software stack for wired or wireless communications with other devices. Memory 650 may include graphical user interface instructions 656 to facilitate graphic user interface processing; sensor processing instructions 658 to facilitate sensor-related processing and functions; phone instructions 660 to facilitate phone-related processes and functions; electronic messaging instructions 662 to facilitate electronic-messaging related processes and functions; web browsing instructions 664 to facilitate web browsing-related processes and functions; media processing instructions 666 to facilitate media processing-related processes and functions; GNSS/Location instructions 668 to facilitate generic GNSS and location-related processes and instructions; and applications 670 that implement the processes described in reference to FIGS. 2-5. Memory 650 further includes other application instructions 672 including but not limited to instructions for applications that utilize outputs of system 100 shown in FIG. 1.

Each of the above identified instructions and applications can correspond to a set of instructions for performing one or more functions described above. These instructions need not be implemented as separate software programs, procedures, or modules. Memory 650 can include additional instructions or fewer instructions. Furthermore, various functions of the mobile device may be implemented in hardware and/or in software, including in one or more signal processing and/or application specific integrated circuits.

While this specification contains many specific implementation details, these should not be construed as limitations on the scope of any inventions or of what may be claimed, but rather as descriptions of features specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable sub combination. Moreover, although features may be described above as acting in certain combinations and even initially claimed as such, one or more features from a claimed combination can in some cases be excised from the combination, and the claimed combination may be directed to a sub combination or variation of a sub combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

As described above, some aspects of the subject matter of this specification include gathering and use of data available from various sources to improve services a mobile device can provide to a user. The present disclosure contemplates that in some instances, this gathered data may identify a particular location or an address based on device usage. Such personal information data can include location-based data, addresses, subscriber account identifiers, or other identifying information.

The present disclosure further contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. For example, personal information from users should be collected for legitimate and reasonable uses of the entity and not shared or sold outside of those legitimate uses. Further, such collection should occur only after receiving the informed consent of the users. Additionally, such entities would take any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices.

In the case of advertisement delivery services, the present disclosure also contemplates embodiments in which users selectively block the use of, or access to, personal information data. That is, the present disclosure contemplates that hardware and/or software elements can be provided to prevent or block access to such personal information data. For example, in the case of advertisement delivery services, the present technology can be configured to allow users to select to “opt in” or “opt out” of participation in the collection of personal information data during registration for services.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, content can be selected and delivered to users by inferring preferences based on non-personal information data or a bare minimum amount of personal information, such as the content being requested by the device associated with a user, other non-personal information available to the content delivery services, or publicly available information.

What is claimed is:

1. A method comprising:
determining, with at least one processor, heartbeat timestamps from a heartrate signal;
obtaining an electroencephalography (EEG) signal from an EEG sensor;
aligning, with the at least one processor, the EEG signal with the heartbeat timestamps;
collaborative filtering, with the at east one processor, the beat-aligned EEG signal; and
determining, with the at least one processor, an approximation of an electrocardiography (ECG) signal based the filtered beat-aligned EEG signal.

2. The method of claim 1, wherein the beat-aligned EEG signal is a two-dimensional (2D) matrix.

3. The method of claim 2, wherein the ECG signal is determined based on a low-dimensional matrix approximation of the filtered beat-aligned EEG matrix.

4. An apparatus comprising:
at least one processor;
memory storing instructions that when executed by the at least one processor, cause the at least one processor to perform the method of claim 1.

5. A computer-readable storage medium having instructions stored there that when executed by one or more processors of a wearable device, perform the method of claim 1.
